(19) United States
(12) Patent Application Publication
(10) Pub. No.: US 2025/0262161 A1
(43) Pub. Date: Aug. 21, 2025

(54) COMPOSITION, METHOD FOR ITS PREPARATION AND ITS USE FOR INCREASING THE SOLUBILITY OF ACTIVE SUBSTANCES

(71) Applicant: Philipps-Universität Marburg, Marburg (DE)

(72) Inventor: Cornelia Keck, Marburg (DE)

(73) Assignee: Philipps-Universität Marburg, Marburg (DE)

(21) Appl. No.: 18/702,938

(22) PCT Filed: Oct. 19, 2022
(86) PCT No.: PCT/EP2022/079121

§ 371 Date: Apr. 19, 2024

(30) Foreign Application Priority Data
Oct. 21, 2021 (EP) 21203972.1

(51) Int. Cl. A61K 9/20; A61K 31/12; A61K 47/46

(52) U.S. Cl. CPC A61K 9/2068; A61K 9/2013; A61K 31/12; A61K 47/46

(57) ABSTRACT

The present application relates to a composition for increasing the solubility of an active substance, comprising crushed shell material, on or in which the active substance is sorbed. Furthermore, a method for producing the composition as well as its use are described

The present invention relates to a composition, a method for its preparation and its use for increasing the solubility of active substances.

Active substances can only be absorbed by the body if they are available dissolved at the site of absorption. In many cases, this is a problem today, i.e., many substances cannot be absorbed by the body, or only inadequately, due to poor solubility. In pharmacy, this affects up to 90% of all newly synthesized active ingredients. Strategies must therefore be found to make such active ingredients readily soluble.

To date, there are various strategies to improve solubility, including so-called nanocrystals, solid solutions, mixed crystals, porous materials, such as porous silica, etc.

Nanocrystals are the most commonly used formulation strategies to date regarding the number of products on the market. Disadvantage here is that the solubility can only be increased by a factor of about 2-3.

Mixed crystals and solid solutions achieve good increases in solubility. However, these are not universal strategies, as individual formulations must be developed for each individual active ingredient. This is time-consuming and cost-intensive. The additives used for this purpose are often expensive and less environmentally friendly. This applies to production and also to biodegradability. The number of market products is correspondingly small.

Porous materials are comparatively to be regarded as a relatively “young” technology for increasing solubility. The principle recognized to date for increasing solubility is the incorporation of active ingredients into nanoscale pores (mesopores) in amorphous form. The increase of solubility with this method is very good (usually 3-10 times). The disadvantage of the method is the use of silicate particles, which cannot be degraded by the body (and nature) and which-depending on their origin—may also contain higher amounts of aluminium (suspected promotion for the manifestation of Alzheimer's disease). The production of the porous silicate particles is still expensive and not environmentally friendly. Producing tablets of the porous materials is only possible with the addition of various excipients, i.e., the amount of added silicate particles is <10%, so that the total amount of active ingredient (loaded into the silicate particles) is also correspondingly small.

M. M. Than (Mahidol University Journal of Pharmaceutical Sciences 2012; 39 (3-4), 32-38) describes the utilization of eggshell powder as excipient in fast and sustained release acetaminophen tablets. The active substance, in this case acetaminophen, is only physically mixed with the eggshell powder, but it is not sorbed thereto.

The technical object underlying the present invention is seen in providing particles, a method for its production and its use that are made of harmless and preferably biodegradable materials (both in the body and in the environment), that can be produced in a cost-effective and environmentally friendly manner, from renewable raw materials and that also offer a high loading capacity for the active ingredients in the final formulation.

This object is solved by the independent claims. Preferred embodiments are defined in the dependent claims.

According to the present invention, there is provided a composition for increasing the solubility of an active substance, comprising crushed shell material, on or in which the active substance is sorbed.

Sorption as used herein is a collective term for processes that lead to an accumulation of a substance within a phase or at an interface between two phases. The enrichment within a phase is more precisely called absorption, that at the interface adsorption. Accordingly, the term “sorbed” as used according to the present invention encompasses absorption, adsorption as well as a mixture of both.

In one embodiment, the composition according to the present invention is obtainable by dissolving the active substance in a solvent, which is different from a solvent in which the solubility of the active substance shall be increased, in order to obtain a solution, mixing the solution with the crushed shell material and optionally removing at least a part of the solvent, in which the active substance is dissolved. The detailed process steps are explained in more detail in the following when the method according to the present invention is described so that in so far it is referred to the following. It is pointed out that it is not clear whether the process of sorbtion of the active substance to the crushed shell material is adsorption, absorption or a mixture of both so that an adequate way to describe the composition is the method for its preparation.

Furthermore, the active substance can be hardly soluble or insoluble in one solvent but be readily soluble in another solvent. For carrying out the process for preparing the composition according to the present invention, it goes without saying that a solvent is chosen that dissolves the active substance but this is not the solvent in which the solubility of the active substance shall be increased.

The term “active substance” means any substance which has a desired effect. In particular, active substances are substances that have a specific effect or cause a specific reaction in an organism. In one embodiment, the active substance is an active pharmaceutical ingredient. Usually, the solubility of a substance shall be increased which has a poor solubility, in particular the physiological environment of a human or animal body. The active substance as used in the composition according to the present invention may be sparingly soluble, poorly soluble, very poorly soluble or practically insoluble, wherein sparingly soluble means 30 parts to 100 parts, poorly soluble means 100 parts to 1 000 parts, very poorly soluble means 1 000 parts to 10 000 parts and practically insoluble means more than 10 000 parts volume solvent for 1 mass part substance at 15° C. to 25° C.

For the purposes of the present invention, a shell material means the outer covering or husk of a seed, fruit or nut, and the shell of mollusks. In one embodiment, the crushed shell material can be selected from crushed egg shell and/or crushed nutshell. The term “and/or” means that also a mixture of crushed egg shell and crushed nut shell can be employed.

The composition according to the present invention can contain crushed eggshells. As egg for obtaining the eggshells any egg can be used, but hen's eggs are preferred since they are readily available, and pharmaceutically acceptable because they are used as food product. The shell material can also be from a nut, in particular a walnut, which is also readily available. The term “crushed” indicates that the shell material used in the composition according to the present invention has a smaller size compared to the shell material naturally appearing. Crushed eggshells are commercially available for the nutrition of dogs and cats.

In one embodiment, the crushed shell material is present as a ground, powdered, micronized or nanonized material. During grinding, the shell material is crushed (with a grinder) by grating and crushing. A powder as used herein is a (nearly) dust finely crushed, pulverized, ground substance. Micronization is the significant reduction of the average particle size, wherein the usual particle sizes between 100 μm and 1000 μm are reduced to a spectrum of 2 μm to 200 μm. In the nanonized material the crushed shell material is present in the nanoscale. These are usual techniques for crushing materials. The skilled person knows methods and materials to carry them out. The crushing in the nanoscale can be carried out in the presence of a liquid.

In one embodiment, the crushed shell material is present as a coarse-grained powder, a medium-fine powder, a fine powder, or a very fine powder. The fineness of powders can be characterized by specifying one or two sieve numbers, determining the percentage m/m of material passing through each sieve. The sieve table of Ph. Eur. lists 18 standardized sieves; as sieve numbers it uses the clear square mesh sizes in μm: 11 200; 8 000; 5 600; 4 000; 2 800; 2 000; 1 400; 1 000; 710; 500; 355; 250; 180; 125; 90; 63; 45; 38. When two sieves are used, the sieve size (A) which is passed by equal or more than 95% of the samples and a second sieve size (B) which is passed by equal or less than 40% are determined. Based on this method, Ph. Eur. defines four grain classes:

| Grain class | Sieve (A) | Sieve (B) |
|---|---|---|
| coarse-grain | 1400 | 355 |
| medium fine | 355 | 180 |
| fine | 180 | 125 |
| very fine | 125 | 90 |

In one embodiment, the crushed shell material is loaded with from 0.1% to 100% of the maximum loading capacity of the crushed shell material with the active substance.

In a further embodiment, the composition is present in suspensions, creams, gels, ointments, pastes, capsules, granules, pellets, tablets, drops, sprays, suppositories, lozenges, patches, pens. These are usual formulations in the field of medicaments. The skilled person knows the methods and the materials for obtaining these formulations, i.e. to provide the composition according to the present invention in these formulations.

In one embodiment, the composition according to the present invention further contains a compound selected from the group consisting of C3-C10 trihydroxy compounds, for example glycerol, and/or an acid, for example citric acid. It has been surprisingly found that the addition of these further compounds increases the passive penetration of the active substance for example the penetration through the skin.

The present invention has shown that the composition according to the present invention can be obtained by simply adding crushed shell material, for example crushed egg shell and/or crushed nutshell (ground, powdered, micronized or nanonized) to active ingredient solutions. After removing, for example evaporation, of the solvent, the active ingredient accumulates in or on the shell particles, increasing its solubility by up to 5-fold. The increased solubility leads to improved active ingredient penetration. Poorly soluble active ingredients can be made readily bioavailable with the technology. Fields of application are oral application, topical application. For application in or on humans, animals or other surfaces (e.g., for application of poorly soluble fertilizers, pesticides, surface disinfection, etc.).

Eggshells are approved as foodstuffs and therefore harmless in their application. They can be obtained from renewable raw materials and, as a waste product, are particularly readily and inexpensively available. Any type of active substance can be loaded onto and/or into shell materials. It is therefore a universal method. Shell material loaded with active substances (or unloaded) can either be applied directly as powder or incorporated into liquid or semi-solid formulations, filled into capsules or simply compressed into tablets. Studies show that the tablets obtained in this way meet the regulatory requirements of the European Pharmacopoeia. Eggshell formulations can thus be used as a new, cost-effective and particularly environmentally friendly method for improving the efficacy of poorly soluble substances in a wide range of sectors (pharmaceuticals, healthcare, food, cosmetics, textile industry, paints and coatings, agriculture, etc.). The principle is simple, but smart, i.e., easy to produce, applicable, and therefore has a high utility.

The present invention further relates to a method for preparing a composition for increasing the solubility of an active substance, in particular the above described composition according to the present invention, comprising crushed shell material, on or in which the active substance is sorbed, wherein the composition is obtained by dissolving the active substance in a solvent, which is different from a solvent in which the solubility of the active substance shall be increased, in order to obtain a solution, mixing the solution with the crushed shell material and optionally removing at least a part of the solvent, in which the active substance is dissolved.

The desired active substance is dissolved in a suitable solvent and the solution is applied to crushed shell material. As pointed out above, the active substance can be soluble in one solvent but insoluble in another solvent. For preparing the composition, a solvent is chosen which dissolves the active substance because otherwise no sorption is possible. Such a suitable solvent furthermore can be pharmaceutically acceptable. Typical suitable solvents are ethanol or oils. The solvent can be evaporated. Stirring and/or heat and/or an extraction system for the solvent can be used to accelerate the process. After a nearly complete optional removal of the solvent, loaded shell materials are present as a powder, which can then be used directly or further processed into other formulations (suspensions, creams, gels, ointments, pastes, capsules, granules, pellets, tablets, drops, sprays, suppositories, lozenges, patches, pens, etc.).

By sorping the active substance in or on the eggshells, the solubility of the active ingredient is increased, resulting in improved passive diffusion (bioavailability). The active substance can be released in a sustained manner, so that a long application is possible. Surprisingly, it was found that the addition of acid and glycerol to the composition boosts passive penetration.

In one embodiment, the removing of at least a part of the solvent is carried out by applying heat and/or reduced pressure. Generally, it is suitable but not necessary to completely remove the solvent. The solvent, in particular in small amounts, can be still present as long as it has no negative effect on the further use of the composition according to the present invention.

In one embodiment, the mixture of solvent and crushed shell material is stirred. This stirring can be carried out before and/or during the solvent is removed. Stirring means to bring the components of a liquid into circular motion in order to mix them uniformly.

As pointed out above, the composition according to can be used for preparing suspensions, creams, gels, ointments, pastes, capsules, granules, pellets, tablets, drops, sprays, suppositories, lozenges, patches, pens.

The composition according to the present invention can be used for pharmaceuticals, healthcare, food, cosmetics, textile industry, paints and coatings, agriculture (for example for fertilizers), depending on the kind of the active substance.

The invention will be further illustrated by referring to the following examples and figures. It is pointed out that the examples and figures are intended for illustrating the invention but they shall not be construed to restrict the invention thereto.

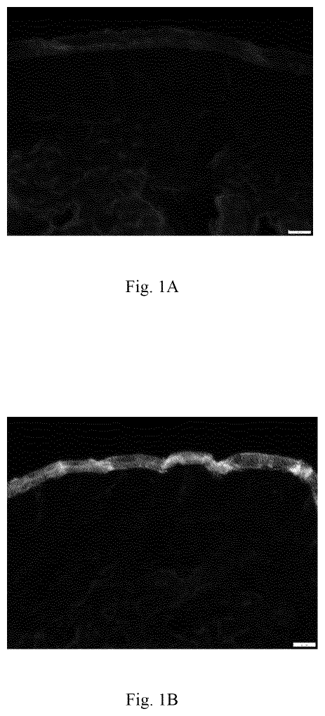

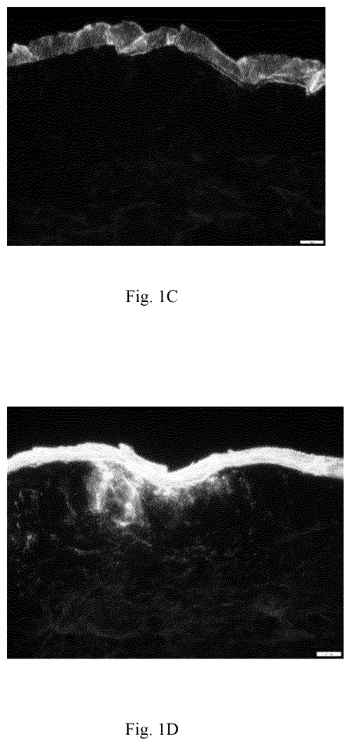

FIG. 1 shows fluorescence microscopy images of skin sections of untreated skin (A), treated with bulk material (B), physical mixture (C) and composition according to the present invention (D). 200× magnification, size scale corresponds to 50 μm.

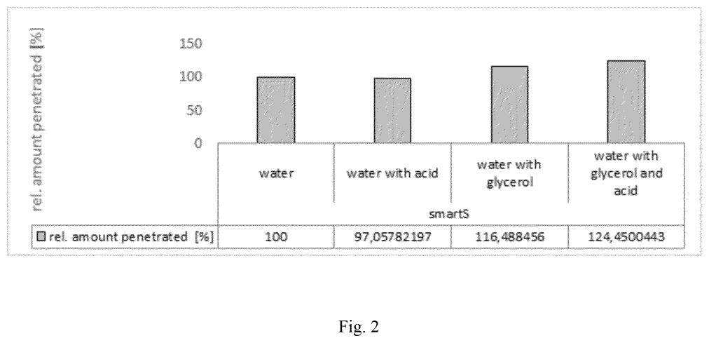

FIG. 2 shows the influence of additives on the penetration of curcumin. The addition of glycerol, as well as acid and glycerol significantly increased the penetration of curcumin into the skin (one-factor ANOVA with Bonferroni-Holm corrected post-hoc analysis).

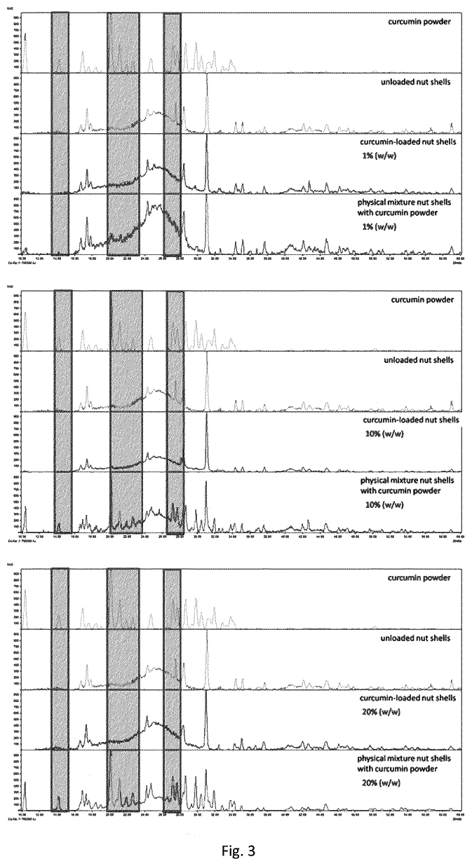

FIG. 3 shows X-ray diffraction patterns of pure curcumin raw powder, unloaded nutshells, curcumin-loaded nutshells and physical mixtures. A: loading with 1% curcumin extract, B: loading with 10% curcumin extract C: loading with 20% curcumin extract.

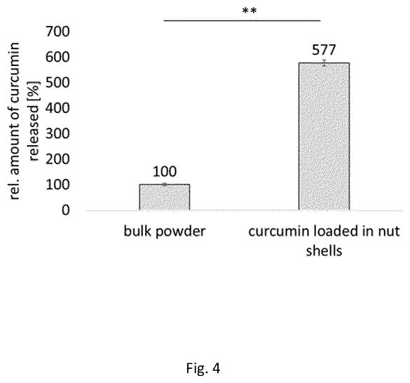

FIG. 4 shows the release of curcumin in water. **-p<0.01 (Welch-Test, n=3).

### EXAMPLE 1 SOLUBILITY

Curcumin is a poorly soluble substance and served as a surrogate for very poorly soluble active ingredients in this experiment. The aim was to demonstrate that the incorporation of poorly soluble substances into eggshells can improve their solubility. Curcumin was first incorporated into eggshell flour. Two different methods were used for this purpose. Method 1—Curcumin was dissolved in ethanol and the solution was placed on eggshells. Ethanol was evaporated without stirring (HP). Method 2—Curcumin was dissolved in ethanol and the solution was placed on the eggshells. Ethanol was evaporated with stirring (R). For comparison, curcumin was mixed with eggshell meal (physical mixture=PM) and added to water as a pure substance (bulk). From all samples, the amount of dissolved curcumin was measured. The results show that when curcumin was incorporated into the eggshells, the aqueous supernatants turned strongly yellow, i.e., curcumin was effectively dissolved in the water phase. No discolouration of the water phase occurred in the physical mixture and the pure substance-thus, no dissolution of curcumin occurred here. The dissolved amount of curcumin was determined at different time points by UV/Vis measurement. For method 1, the solubility for curcumin was about 100 times higher than the comparative solution with pure curcumin. For method 2, the solubility was approximately 50 times higher.

The loaded eggshells contained 10% (w/w) curcumin, i.e., 90% eggshell and 10% curcumin. The physical mixture contained similar amounts.

### EXAMPLE 2—BIOLOGICAL EFFECTIVENESS

The aim was to show that passive penetration of poorly soluble active ingredients, i.e. their biological availability, can be improved by loading in eggshells. The dermal availability was tested in the ex-vivo pig ear model. Curcumin loaded eggshells (method 1 of example 1) were mixed with water in a 1:1 ratio and applied to fresh and uninjured pig skin. The amount of active ingredient penetrated was analysed after 4 h of penetration. Pure curcumin powder dispersed in water and the physical mixture served as comparison. Curcumin loaded in crushed eggshell could effectively penetrate into the skin and showed transdermal penetration. Pure curcumin powder and the physical mixture penetrated slightly into the upper layers of the skin and showed no transdermal penetration (FIG. 1A to 1D).

### EXAMPLE 3—OPTIMIZED BIOLOGICAL EFFECTIVENESS THROUGH ADDITIVES

Surprisingly, it was found that the addition of glycerol and acid (citric acid) to the curcumin-loaded eggshell increased the biological availability of curcumin again by about 25% (FIG. 2).

### EXAMPLE 4—USE FOR ORAL APPLICATION

Eggshell meal was manually compressed into tablets. It was shown that tablets of pharmaceutical quality—without the addition of further excipients—can be produced from eggshell meal by direct tableting.

### EXAMPLE 5—COMPOSITION COMPRISING CURCUMIN AND NUTSHELL

An ethanolic curcumin solution that contained 0.5% (w/v) curcumin extract was added to nutshells (from walnut). The ethanol was evaporated, and the resulting nutshells contained either 1%, 10%, 20% or 40% curcumin extract (w/w). The crystallinity of the curcumin loaded in the nutshells was determined with X-ray diffractometry and the diffractograms obtained were compared to a physical mixture that contained nutshells and curcumin extract raw bulk powder. Raw curcumin extract powder and unloaded nutshells served as controls (FIG. 3). Raw curcumin bulk material showed typical reflexes of crystalline curcumin. The reflexes were also visible in the physical mixtures but disappeared when the curcumin was loaded into the nutshells, indicating that curcumin was loaded into the nutshells in amorphous state (FIG. 3).

The amount of released curcumin after the addition of water was analysed by determining the fluorescence intensity of the supernatant after adding water to the curcumin-loaded nutshells. Curcumin raw powder extract served as control. The released curcumin from the nutshells was 577% when compared to the control (FIG. 4).

1. A composition for increasing the solubility of an active substance, comprising crushed shell material, on or in which the active substance is sorbed.

2. The composition according to claim 1, wherein the composition is obtainable by dissolving the active substance in a solvent, which is different from a solvent in which the solubility of the active substance shall be increased, in order to obtain a solution, mixing the solution with the crushed shell material and optionally removing at least a part of the solvent, in which the active substance is dissolved.

3. The composition according to claim 1, wherein the active substance is an active pharmaceutical ingredient.

4. The composition according to claim 1, wherein the crushed shell material is crushed eggshell and/or crushed nutshell.

5. The composition according to claim 1, wherein the crushed eggshell is from hen's eggs, and/or wherein the crushed nutshell is from a nut, in particular walnut.

6. The composition according to claim 1, wherein the crushed shell material is present as a ground, powdered, micronized or nanonized material.

7. The composition according to claim 1, wherein the crushed shell material is present as a coarse-grained powder, a medium-fine powder, a fine powder, or a very fine powder.

8. The composition according to claim 1, wherein the crushed shell material is loaded with from 0.1% to 100% of the maximum loading capacity of the crushed eggshell with the active substance.

9. The composition according to claim 1, wherein the composition is present in suspensions, creams, gels, ointments, pastes, capsules, granules, pellets, tablets, drops, sprays, suppositories, lozenges, patches, pens.

10. The composition according to claim 1, further containing a compound selected from the group consisting of C3-C10 trihydroxy compound and/or an acid.

11. A method for preparing a composition for increasing the solubility of an active substance, comprising crushed shell material, on or in which the active substance is sorbed, wherein the composition is obtained by dissolving the active substance in a solvent, which is different from a solvent in which the solubility of the active substance shall be increased, in order to obtain a solution, and mixing the solution with the crushed shell material.

12. The method according to claim 11, wherein at least a part of the solvent is removed, in which the active substance is dissolved, wherein said removing of at least a part of the solvent is preferably carried out by applying heat and/or reduced pressure.

13. The method according to claim 11, wherein the mixture of solvent and crushed shell material is stirred.

14. The use of the composition according to claim 1 in suspensions, creams, gels, ointments, pastes, capsules, granules, pellets, tablets, drops, sprays, suppositories, lozenges, patches, or pens.

15. The use of the composition according to claim 1 for pharmaceuticals, healthcare, food, cosmetics, textile industry, paints and coatings, or agriculture.
